(19) United States
(12) Patent Application Publication
(10) Pub. No.: US 2015/0186849 A1
(43) Pub. Date: Jul. 2, 2015

(54) COMPUTER IMPLEMENTED METHOD AND SYSTEM FOR SCHEDULING MOMENT-BASED TASKS ASSOCIATED WITH A MEDICAL TREATMENT AND COMPUTER PROGRAM THEREOF

(71) Applicant: Telefonica Digital Espana, S.L.U., Madrid (ES)

(72) Inventors: Luis CURIEL, Granada (ES); Helio HUETE, Granada (ES); Remko VERMEULEN, Barcelona (ES); Jose VITTONE, Barcelona (ES)

(73) Assignee: Telefonica Digital Espana, S.L.U., Madrid (ES)

(21) Appl. No.: 14/141,117

(22) Filed: Dec. 26, 2013

(51) Int. Cl. G06Q 10/10; G06Q 50/22

(52) U.S. Cl. CPC G06Q 10/1095; G06Q 50/22

(57) ABSTRACT

A computer implemented method and system for scheduling moment-based tasks associated with a medical treatment and computer program thereof. The method includes receiving by at least one patient a moment-based list of tasks as a patient's instruction specifying when a number of given tasks should be performed; assigning for each task of said list of tasks an amount of time or time normal zone indicating an optimal period of time to perform a given task; and assigning, for each time normal zone, a previous time zone and a subsequent time zone of the time normal zone indicating that said task also can be done. In case of overlapping of time normal zones and/or previous or subsequent time zones for a given number of tasks of a same type an algorithm is executed in order to resolve the conflicts between said overlapped tasks.

### FIELD OF THE INVENTION

The present invention generally relates to scheduling tasks. Particularly, present invention relates to a computer implemented method and system for scheduling moment-based tasks associated with a medical treatment and computer program thereof.

### BACKGROUND OF THE INVENTION

Patients are required to do tasks like taking their bio measurements, do exercise, take a medicine or answer a survey on a regular base to monitor and manage their disease better. Taking these measurements is done at various moments of the day.

Current systems treat the tasks that a patient needs to accomplish as if they were events or to-do items in a calendar. As a result, patients can get confused about what to do when the time of the event is up and they have not already accomplished the task.

Actually, most of current systems use some of the current calendar standards that exists nowadays (i.e. iCalendar standard) and, as a result, many patients that, for some reason, do not accomplish the tasks before its time is up, just decide to ignore the task because they have no information about what to do in that scenarios. There are also other patients that just decide to accomplish the task; no matter how long it is since the task time is up.

Actual systems implement agenda tasks as date/time configurations. Most of the implementations allow a user to add additional information as interval, periodicity and reminders, but they are also based on time (hours, minutes, and seconds) and are very hard to specify for every patient.

Another problem with current solutions is that they don't take into account previous daily tasks already accomplished by the patient.

For instance, the Outlook Calendar is an example implementation of specific events based on calendar with time range, repetitions, reminders, etc., However, there is no abstract concept of time, nor to create a dynamic range based on the country, culture or taking into account previous events.

iCalendar, on another hand, it is a standard (RFC 5546) for exchanging calendar information. The standard is also known as “iCal” due to Apple program name, which was the first application to implement it. The problem with this format is that it ends up defining a single event at a specific time, which even allowed ranges are not dynamic or adaptable by country and culture.

U.S. Pat. No. B2-8,068,015 discloses a medication dispensing system for use with a medication dispenser that includes a physiological module and an ingestion module. The physiological module receives information regarding time-based medication intake, event-based medication prescription, and sensory input. Once processed, that information is redirected to the ingestion module and it is used to delay or accelerate the dispensing of medication. On contrary of said patent, the present invention solution describes a method to enrich agenda events with the information provided by day moments and a method to adapt those moments to different cultures and personal habits of the patient. Moreover, present invention provides a mechanism in order tasks of the same type, for instance taking two bio-measurements, not been overlapped.

That is, today's calendar systems are based on static timestamps. When a user wants to schedule an event or a task using a calendar system, s/he is asked to enter the exact time when the event or task is intended to happen. S/he can be asked as well for the exact time when the event or task is going to end and the exact event or task duration. This kind of constraints makes the event or task scheduling an artificial process that, in many times, does not reflect accurately the event or task nature.

### SUMMARY OF THE INVENTION

Present invention solves the above-discussed deficiencies of the tasks scheduling problem for patients using moments instead of times. Moreover, present invention takes into consideration already accomplished tasks.

Therefore, in accordance with a first aspect the invention provides a computer implemented method for scheduling moment-based tasks associated with a medical treatment, comprising receiving by at least one patient a moment-based list of tasks as a patient's instruction specifying when a number of given tasks should be performed. On contrary of the known proposal the method comprises: assigning, for each task of said list of tasks, an amount of time or time normal zone indicating an optimal period of time to perform a given task; and assigning, for each time normal zone, a previous time zone and a subsequent time zone of the time normal zone indicating that said task also can be done.

The method, in case of overlapping of time normal zones for a given number of tasks of a same type, e.g. two bio measures, performs following steps: creates a time single zone as union of said normal zones involved in the overlapping encompassing all the coverage of time of said normal zones; divides said time single zone by said number of tasks; and assigns the time period result of said dividing to each normal zone of said tasks preserving the normal temporal order.

Moreover, in case of overlapping of any of said previous or subsequent time zones the method creates a time single zone as the intersection of the overlapped time zones; divides said time single zone by said number of tasks; preserves said normal time zone for each of the tasks, and assigns the time period result of said dividing to said previous or subsequent time zone of each of the tasks.

As a preferred option of the present invention, the time normal zone defines an optimal moment to perform a task. This optimal moment is based on daily habits of said patient including at least a given period in the morning, before or after breakfast, before or after lunch, before or after dinner, in the afternoon, before going to bed, etc.

According to different embodiments, the previous and subsequent time zones can have the same or a different duration.

In an embodiment, the moment-based list of tasks is received by displaying it on a screen of a computing device. Furthermore, the moment-based list of tasks can be stored in a database of a computing device.

In another embodiment, a task can be removed, either automatically or manually by the patient, of said list of tasks upon completion thereof.

In yet another embodiment, an alarm can be blow up in a computing device of said patient before starting said time normal zone. For instance, it can be blow up 5 minutes before said starting.

In accordance with a second aspect there is provided a computer implemented system for scheduling moment-based tasks associated with a medical treatment, comprising means for at least transmitting to at least one patient a moment-based list of tasks as a patient's instruction specifying when a number of given task should be performed. The system of the second aspect further includes:

first means for assigning, for each task of said list of tasks, an amount of time or time normal zone indicative of an optimal period of time to perform a given task;

second means for assigning, for each time normal zone, a previous time zone and a subsequent time zone indicative that said task can be done; and

means for performing an algorithm in case of an overlapping of time normal zones for a given number of tasks and/or in case of an overlapping of any of said previous or subsequent time zones.

In the system in case of said overlapping being in the time normal zone for a given number of tasks of a same type said algorithm comprises: creating a time single zone as union of said normal zones involved in the overlapping encompassing all the coverage of time of said normal zones; dividing said time single zone by said number of tasks; and assigning the time period result of said dividing to each normal zone of said tasks preserving the normal temporal order.

In addition, in case of said overlapping being in said previous or subsequent time zones said algorithm comprises: creating a time single zone as the intersection of the overlapped time zones; dividing said time single zone by said number of tasks; preserving said normal time zone for each of the tasks, and assigning the time period result of said dividing to said previous or subsequent time zone of each of the tasks.

The means for transmitting are further configured to receive a feedback upon completion of any of said tasks.

The system of the second aspect is adapted to implement the method of the first aspect.

The subject matter described herein can be implemented in software in combination with hardware and/or firmware, or a suitable combination of them. For example, the subject matter described herein can be implemented in software executed by a processor.

According to a third aspect there is also provided a computer program product comprising a computer-readable medium having instructions stored thereon, the instructions being executed by one or more processors and the instructions comprising instructions for carrying out a method for inter-working different wireless technologies according to the steps of claim 1.

Present invention adapts better to the medical requirements on when to fulfil tasks as it adapts better to them as is built in flexibility. So, likelihood that patients perform tasks will be higher. This as a result leads to a better medical adherence which has proven to lower health risks.

### BRIEF DESCRIPTION OF THE DRAWINGS

The previous and other advantages and features will be more fully understood from the following detailed description of embodiments, with reference to the attached, which must be considered in an illustrative and non-limiting manner, in which:

FIG. 1 illustrates the provided software application system for scheduling moment-based tasks associated with a medical treatment, according to some embodiments of the present invention.

FIG. 2 illustrates two examples of different types of tasks with different moment-based configuration.

FIG. 3 illustrates the proposed algorithm or process executed in case of overlapping of two tasks of the same type in a normal zone.

FIG. 4 illustrates the proposed algorithm or process executed in case of overlapping a before zone or in an after zone.

FIG. 5 illustrates the basic operation of the system platform in accordance with an embodiment.

### DETAILED DESCRIPTION OF SEVERAL EMBODIMENTS

Present invention provides a moment-based calendar system for scheduling tasks that, in contrast of today's calendar systems, can adapt the static and inflexible timestamps in order to describe, in a more accurately way, the nature of many daily events or tasks. Due to different cultures and personal habits, events and tasks timing can be modified in order to adapt better to what the user of the calendar system needs to schedule.

In some embodiments, the provided moment-based task system can warn, via an alarm, the patient when the event or task is about to start or end.

Present invention is integrated within a software application system or Tele-Health system. This Tele-Health system is made out of two different parts, a platform and the gateway. The platform collects all the information and provides tools to visualize data, and the gateway collects information from the patient and from the sensors and forwards said information to the Platform. The platform allows including in the patient calendar moment-based tasks that adjust to what the doctor expects when defining the task.

The gateway can be either a final user computing device (such a cell phone, a tablet, a personal computer, etc.) or a M2M device designed to redirect the data from/to the platform to/from a final user computing device.

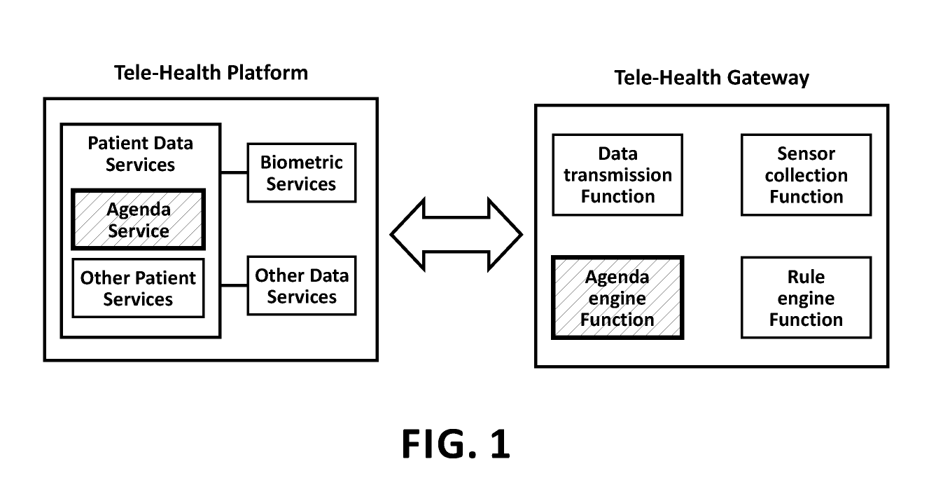

FIG. 1 illustrates the provided Tele-Health system. The Agenda Service unit/means and the Agenda engine Function unit/means provide the main characteristic of the present invention.

The Agenda Service unit/means (in the platform side) allows the doctors to define moment-based tasks for patients. This service is provided with detailed information about the relation between moments and key hours for every culture that it supports. The platform allows to add new custom moments, as many as the admin wants to define. The platform also allows the tasks and the moment/hour information to be downloaded from the patients' gateways.

When a doctor defines a task, s/he also defines the moment that this task will be associated to. Preferably, these moments will be in the morning, before breakfast, after breakfast, before lunch, after lunch, in the afternoon, before diner, after diner, in the evening and before going to bed, among others . . . The doctor is not limited to a list of moments and can add his/her own moments. In addition, the doctor can also define types of tasks, and assigns them the desired margins (when the task can be completed) and reminders.

User Profiles and Programs

These moments are translated to a certain key hour depending on each country/user. These values are configurable as to adapt to changes in habits. For example “After diner” in Spain can has a key hour 22:00 while in the UK said key hour is at 20:00. This is the concept of profile in the platform.

Several profiles can be defined, so assigning moments to patients can be done automatically just choosing a profile for every patient. The profile contains the definition of the moments and their times.

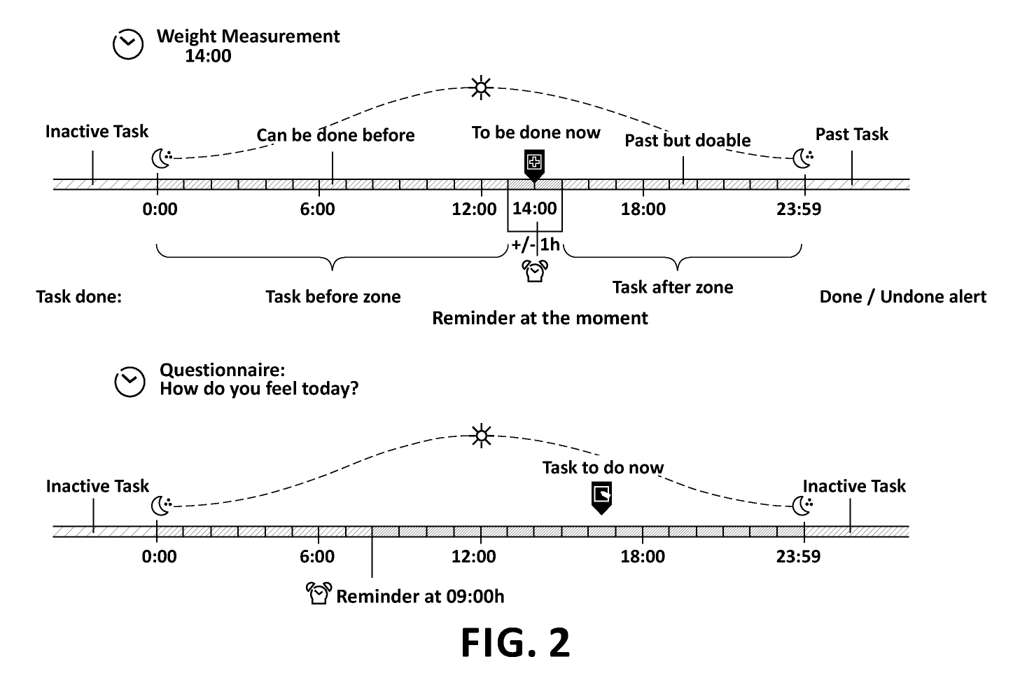

Within the profile, a moment has a configurable duration that defines a time range when the patient can perform the task normally (OPT), before (B) and after (A), as illustrated in FIG. 2, that range a grace period that can be configured in which the patient can still perform the task. This range (margins) depends on the type of the task the doctor has defined.

In addition, a task can be scheduled with N configurable reminders. These reminders can be used to alert the patient when the chosen moment is near in time or even when it is about to end and those reminders can be included in the profile.

The program is the set of tasks that a patient needs to complete depending on their condition, for example, a program for an asthmatic patient, high blood-pressure patient, etc.

A program contains a set of tasks that all patients with the same condition must complete every day. This information will be used to fill the patient agenda, and when mixed with the profile, the agenda is customized for every profile. So, with the proposed method, it is very simple to assign patients to a profile and to a program resulting in a customized agenda for every patient.

The “Agenda engine Function” unit/means will allow the patients to view their moment-based agenda tasks in an understandable way. It downloads the configuration of the profile and the program from the remote platform using (but not limited to) WiFi, wired, 3G or any other kind of Internet connection access. The Agenda engine Function unit/means can translate the moments of the tasks to a key hour by using the patient culture. The moment has a predefined base time. Then this time can be overridden by the profile (patient culture) and then localised using local time of patient or user (profile) or gateway.

Moreover, the Agenda engine Function unit/means can adjust the tasks key hour by using the patient time zone (in the case the patient is in a different time zone than the person creating the task). Also, can resolve overlapping issues in patients agenda (in the case two or more tasks of the same type are overlapped), show the agenda to the patient, warn the patient when a task time is close to start (e.g. 5 minutes before starting), remind the patient in case the doctor has defined some reminder for a task, highlight those tasks that are about to finish, take into account already accomplished tasks to show detailed and updated information to the patient. To know when a task is completed, the patient can mark it as done manually or if an automatic device is present that sends information to the gateway, it will get marked as done automatically.

In addition, the Agenda engine Function unit/means can inform the patient about what tasks have been performed, what tasks are about to start and what tasks need to be performed in the near future. A finished task can affect how the rest of the tasks are shown to the patient, especially when they are of the same type and they overlap. It can get the information from the remote platform or could be manually included in the gateway by the patient.

FIG. 2 illustrates two examples of different types of tasks with different moment-based configuration. The patient uses the gateway to get the tasks he/she needs to complete at every moment/time. The gateway shows a list of tasks depending on the actual time/moment and shows also the already completed tasks and the next ones when the time is getting close.

Per type of task different ranges are defined for each period: before zone (B), normal zone (OPT) and after zone (A).
Before zone (B): Is the range of time previous to the normal zone or optimum period of time in which a task can be done, considering as valid the performance of the same.
Normal zone (OPT): Is the optimal period of time for performing a task.
After zone (A): Is the range of time afterward the normal zone in which a task can be done, considering as valid the performance of the same.

To mark a task as completed the patient can manually choose it on the gateway and mark it as done or it can be automatically marked when using some electronic device that automatically sends the result to the gateway, for example, but not limited to, wireless weight scales, glucometers, electronic pill dispenser, mobile phones, etc.).

When the task is marked as done it will disappear from the “to do now” list and will appear as already completed or completely disappear depending on patient configuration. When the task is completed automatically, for example by using an electronic wireless computing device, the moment is automatically detected by the system depending on the actual agenda configuration and it is marked as done. If the patient detects that the moment has not been correctly chosen s/he has the ability to modify it to update it to the right moment.

In the case of a task that needs to be performed before and after a normal zone or moment like breakfast, the following method has been designed to make the patient focus on the most important task first. Up till the moment that a patient has performed a task and before a specific moment (breakfast, lunch, and/or dinner) the task to be performed after that moment won't be activated.

When two or more tasks of the same type (bio-measure, questionnaire, meeting, etc.) are overlapped, the present invention by means of an algorithm or process separates the tasks in order to resolve the conflicts between agenda tasks. The object of this process is that there are not tasks of the same type at the same moment of time in the normal area. This process will take into account whether the tasks have already been made or not, but it won't take into account the result of the same.

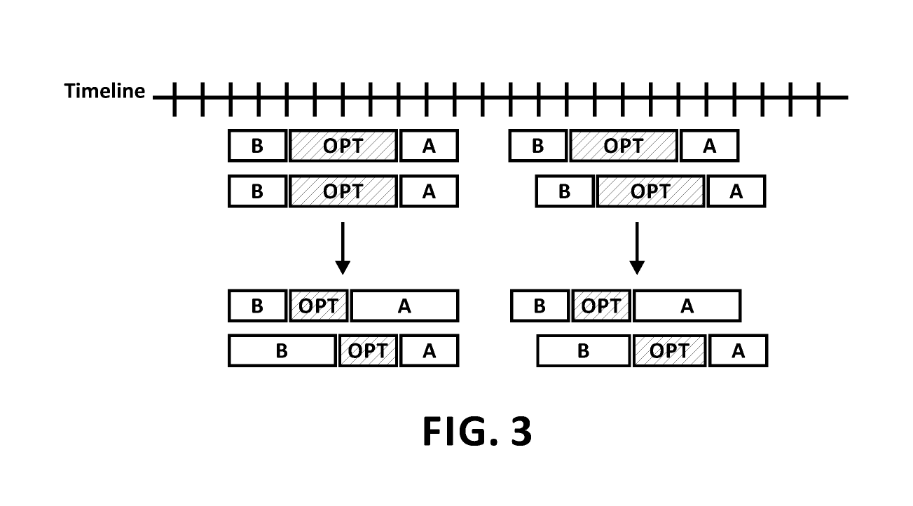

In case of overlapping of two tasks of the same type in a normal zone (OPT), as illustrated in FIG. 3, the invention creates a single zone as the union of the two zones and then assigns to each task the half of the single zone.

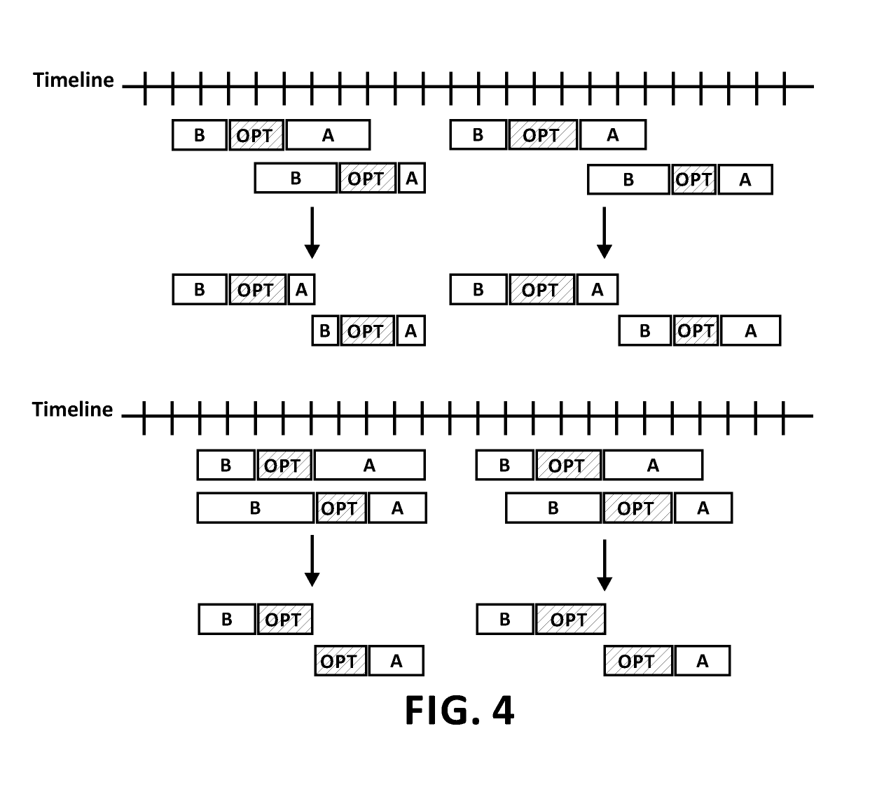

On another hand, in case the overlapping conflict occurs between non normal zone, i.e. it occurs in a before zone (B) or in an after zone (A), as illustrated in FIG. 4, the invention creates a single zone as the intersection of the zones and then, assign to each task the half of the single zone. In this step, the “normal zones” will be never modified so “normal zones” will not be affected.

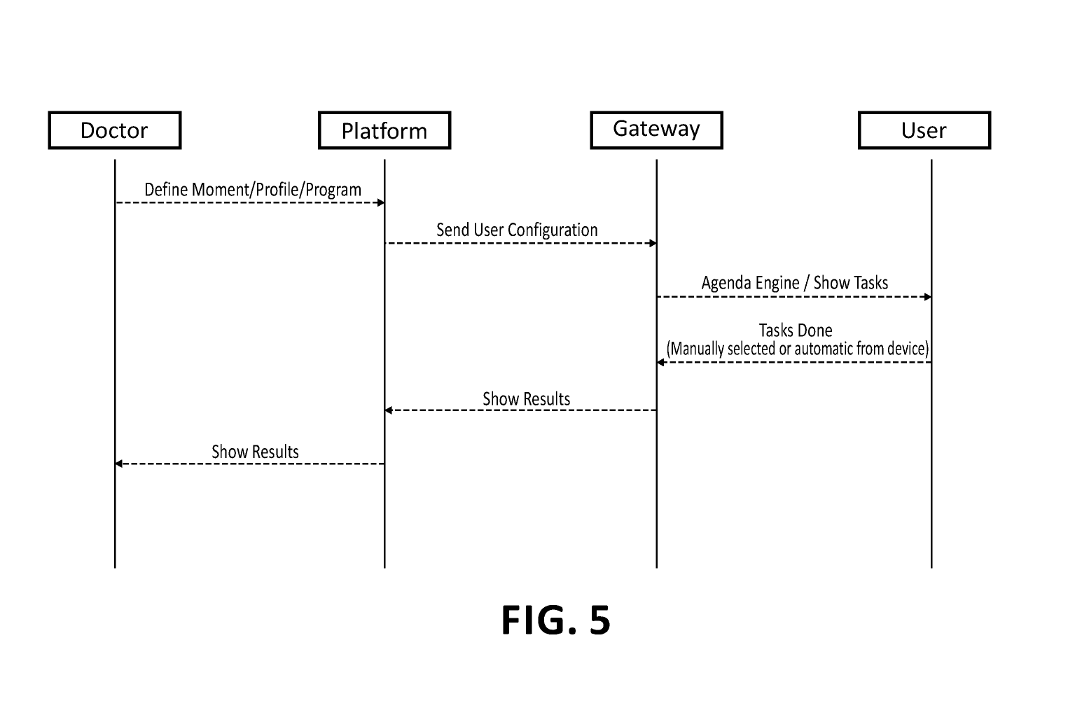

In reference to FIG. 5 it is illustrated the basic operation of the system platform in accordance with an embodiment. Doctor defines the different moments and assigns base times to them, a base time definition is a time range (i.e.: 14:00 h-16:00 h). Then doctor defines tasks, for instance a bio-measurement, a meeting, etc.) and if desired their moment margins (e.g. before and/or after) and/or reminders. Then, doctor creates profiles to specify times to moments. A profile is a set of specific times for moments. For example, a profile for a patient in Spain could be as follows: before breakfast (06:00-8:00), after breakfast (11:00-16:00). This is a specification for the basic times of moments, so this configuration can override default time ranges. If the moment is not overridden here, it uses the base time. At that time, doctor can create programs with said set of tasks. A program is a set of tasks for specific diseases or health programs. (i.e.: program for diabetes patients). Finally, doctor creates a user or patient with a specific profile and program.

Platform sends the data created by the doctor to the gateway and the latter adapts the moments and times to local time zone of the patient. Preferably, all times are defined to be UTC Times (GMT+0), so the time must be adapted to user local time (06:00-08:00 in Spain must be 06:00-08:00 in Canarias as well). The user local time is defined on the patient data modelling of the platform or if not present, using the gateway local time zone. The moment is adapted depending on the profile of the patient if needed.

The gateway executes the overlapping process shown in FIGS. 3 and/or 4 and then shows the result to the patient. So, the patient can perform required tasks and mark them (either manually or automatically) as completed. Upon completion of the required tasks by the patient, the gateway sends the results to the platform therefore the doctor can check the results on the platform.

Some of these tasks can be done simultaneously (for instance the defining of profiles and programs done by the doctor).

With the present invention, a patient can perform a task without querying the agenda, for example take a biomeasure, fill a survey, etc. In this case, if available, the task will be completed automatically and the results will be updated on patient's agenda in the gateway and the results will be automatically send to the gateway and then to the system platform.

The moment defined to perform a task will be automatically detected using an inference algorithm based on actual time and active tasks.

The scope of the present invention is defined in the following set of claims.

1. A computer implemented method for scheduling moment-based tasks associated with a medical treatment, comprising receiving by at least one patient a moment-based list of tasks as a patient's instruction specifying when a number of given tasks should be performed, said method being characterized in that:
for each task of said list of tasks an amount of time or time normal zone is assigned indicating an optimal period of time to perform a given task; and
for each time normal zone a previous time zone and a subsequent time zone of the time normal zone is assigned indicating that said task also can be done;
wherein in case of overlapping of time normal zones for a given number of tasks of a same type comprises performing following steps:
creating a time single zone as union of said normal zones involved in the overlapping encompassing all the coverage of time of said normal zones;
dividing said time single zone by said number of tasks; and
assigning the time period result of said dividing to each normal zone of said tasks preserving the normal temporal order,
and wherein in case of overlapping of any of said previous or subsequent time zones comprises performing following steps:
creating a time single zone as the intersection of the overlapped time zones;
dividing said time single zone by said number of tasks;
preserving said normal time zone for each of the tasks, and
assigning the time period result of said dividing to said previous or subsequent time zone of each of the tasks.

2. The method of claim 1, wherein said tasks of a same type including at least one of a bio measurement, do exercise, take a medication and/or answer a survey on a regular base.

3. The method of claim 1, wherein said time normal zone defines an optimal moment to perform a task based on daily habits of said patient including at least a given period in the morning, before or after breakfast, before or after lunch, before or after dinner, in the afternoon, before going to bed.

4. The method of claim 1, wherein said previous and subsequent time zone comprises a same duration.

5. The method of claim 1, wherein said previous and subsequent time zone comprises a different duration.

6. The method of claim 1, wherein said receiving of said moment-based list of tasks is performed by displaying said moment-based list of tasks on a screen of a computing device.

7. The method of claim 1, wherein a task being automatically removed of said list of tasks upon completion thereof.

8. The method of claim 1, wherein a task being manually removed of said list of tasks upon completion thereof by a communication interface of a computing device of said patient.

9. The method of claim 1, wherein said moment-based list of tasks is stored in a database of a computing device.

10. The method of claim 1, comprising blowing up an alarm in a computing device of said patient before starting said time normal zone.

11. The method of claim 10, wherein said alarm is blow up at least 5 minutes before said starting.

12. A computer implemented system for scheduling moment-based tasks associated with a medical treatment, comprising means for at least transmitting to at least one patient a moment-based list of tasks as a patient's instruction specifying when a number of given task should be performed, said system further comprising:
first means for assigning, for each task of said list of tasks, an amount of time or time normal zone indicative of an optimal period of time to perform a given task;
second means for assigning, for each time normal zone, a previous time zone and a subsequent time zone indicative that said task can be done; and
means for performing an algorithm in case of an overlapping of time normal zones for a given number of tasks and/or in case of an overlapping of any of said previous or subsequent time zones,
wherein in case of said overlapping being in the time normal zone for a given number of tasks of a same type said algorithm:
creating a time single zone as union of said normal zones involved in the overlapping encompassing all the coverage of time of said normal zones;
dividing said time single zone by said number of tasks; and
assigning the time period result of said dividing to each normal zone of said tasks preserving the normal temporal order,
and wherein in case of said overlapping being in said previous or subsequent time zones said algorithm:
creating a time single zone as the intersection of the overlapped time zones;
dividing said time single zone by said number of tasks;
preserving said normal time zone for each of the tasks, and
assigning the time period result of said dividing to said previous or subsequent time zone of each of the tasks.

13. The system of claim 12, wherein said means for transmitting are further configured to receive a feedback upon completion of any of said tasks.

14. A computer program product comprising a computer-readable medium having instructions stored thereon, the instructions being executed by one or more processors and the instructions comprising instructions for carrying out a method for inter-working different wireless technologies according to the steps of claim 1.

15. The method of claim 2, wherein said time normal zone defines an optimal moment to perform a task based on daily habits of said patient including at least a given period in the morning, before or after breakfast, before or after lunch, before or after dinner, in the afternoon, before going to bed.
